(19) United States
(12) Plant Patent Application Publication
(10) Pub. No.: US 2015/0313055 P1
(43) Pub. Date: Oct. 29, 2015

(54) Ornamental perennial peanut 'PP-1'

(50) ;

(71) Applicant: University of Georgia Research Foundation, Inc., Athens, GA (US)

(72) Inventors: Wayne W. Hanna, Chula, GA (US); Brian M. Schwartz, Tifton, GA (US); Ann Rachel Soffes Blount, Quincy, FL (US)

(73) Assignee: University of Georgia Research Foundation, Inc., Athens, GA (US)

(21) Appl. No.: 14/120,105

(22) Filed: Apr. 25, 2014

(51) Int. Cl. A01H 5/00

(52) U.S. Cl. PLT/258

(57) ABSTRACT

The new cultivar of Arachis glabrata ‘PP-1’ is provided. The new and distinct variety has high ornamental value, abundance of yellow orange flowers, dark green color, and low maintenance after establishment. The asexually reproduced cultivar is reliably propagated vegetatively.

### LATIN NAME OF THE GENUS AND SPECIES OF THE PLANT CLAIMED

‘PP-1’ is a vegetatively propagated ornamental perennial peanut cultivar of the genus and species Arachis glabrata.

### VARIETY DENOMINATION

The new Arachis glabrata claimed is of the cultivar denominated ‘PP-1’.

### BACKGROUND OF THE INVENTION

The present invention relates to a new and distinct cultivar of Arachis glabrata herein referred to as ‘PP-1’.

The new Arachis glabrata is a product of a planned research and evaluation program conducted by the Inventors in Tifton, Ga. The objective of the Arachis glabrata research program is to create new plant cultivars with improved commercial qualities. This cultivar is commercially important for its superior ornamental value. These and other qualities are enumerated herein.

Pedigree and history: Annual reports [in the University of Georgia (UGA), Tifton Campus library] by J. L. Stephens, a research agronomist with the U.S. Department of Agriculture at Tifton, Ga. show that Mr. Stephens introduced 12 wild Arachis species and evaluated them in plots on the UGA Tifton Campus from 1952 to 1954. One accession was designated as A. glabrata, three accessions were designated as A. marginata, and the remaining accessions were designated as unknown ‘sp.’ with a number. Observations indicated broad morphological variation within the A. glabrata species, therefore difficulty in establishing species identity without expert assistance. Mr. Stephens wrote in his 1953 annual report that three accessions, Arachis sp. 172223, Arachis sp. 172224, and Arachis sp. DETP 6519 (all unpatented) showed potential for cattle pastures and were vegetatively planted in two-acre pastures in 1954. However, the perennial peanut research was not mentioned in Mr. Stephens' annual reports after 1954; probably due to establishment problems due to vegetative propagation.

‘PP-1’ is a vigorous Arachis glabrata clone volunteering for over 50 years on the University of Georgia, Tifton Campus, Tifton, Ga. on the eastern edge of plots used in 1952 to 1954 to test perennial peanut introductions. ‘PP-1’ probably arose from either an outcross among the A. glabrata accessions being evaluated or a superior genetic recombination from an A. glabrata introduction growing in the 1952 to 1954 plots at Tifton, Ga. A. glabrata flowers profusely and will set occasional seeds. It was discovered by the inventors growing in a natural uniform stand 7 m wide and 123 m long on the eastern edge of the 1952 to 1954 research plots. Growth or spread of the ‘PP-1’ stand has been limited on the east side by herbicides sprayed by the Georgia Department of Transportation along U.S. Route 41 and limited on the west side by cultivation and herbicides sprayed by the University of Georgia. Pictures of ‘PP-1’ were sent to Dr. Charles Simpson at Texas A&M University to help with identification. Dr. Simpson sent the inventor a sample of the Arachis glabrata ‘A42’ variety (unpatented) which was then compared with ‘PP-1’. ‘A42’ has wider leaves (Table 8) and more leaf area per leaf (Table 10) than ‘PP-1’.

A fourteen amplified fragment length polymorphism AFLP study on “Genetic Variability of Cultivated Rhizome Peanut” by Maas, Anderson and Quesenberry [Crop Science 50:1908-1914 (2010)] revealed that ‘PP-1’ is most related to ‘Florigraze’ (unpatented), a broad leaf and tall perennial peanut type plant. ‘PP-1’ is referred to as UGA Experimental in the Mass, et. al. study; the study also included released or soon to be released cultivars and accessions with commercial cultivar potential (the study did not include all of the perennial peanut germplasm in GRIN). Morphologically, ‘PP-1’ is most similar to Natural Resources Conservation Service (NRCS) germoplasm perennial peanut plant ‘Brooksville 68’ (unpatented) commonly referred to as ‘Pointed Leaf’ for which planting stock was not available when we established the 2006 test at Tifton. ‘PP-1’ produces infrequent viable seed. We looked at 40 plants produced from seed of ‘PP-1’ and found that a wide range of morphological types were produced, none with the vigor and persistence of ‘PP-1’.

Asexual reproduction of the new A. glabrata ‘PP-1’ by vegetative propagation from single rhizome sections in a controlled environment in Tifton, Ga. since 2005, has shown that the unique features of this new A. glabrata cultivar are stable and reproduced true to type in successive generations.

### SUMMARY OF THE INVENTION

The following traits have been repeatedly observed and represent the characteristics of a new A. glabrata cultivar ‘PP-1.’ The new cultivar ‘PP-1’ has not been observed under all possible environmental conditions. The phenotype may vary somewhat with variations in, for example, temperature, day-length, light intensity, soil types, and water and fertility levels, without, however, any variance in genotype.

Throughout this specification, color names beginning with a small letter signify that the name of that color, as used in common speech, is aptly descriptive. Color names beginning with a capital letter designate values based upon the R.H.S. Colour Chart, 5^{th} edition published by The Royal Horticultural Society, London, England.

The following traits have been repeatedly observed in Tifton, Ga.; Gainesville and Quincy, Fla. and are determined to be the unique characteristics of the new cultivar ‘PP-1’:

1. ‘PP-1’ reaches a height of about 20 cm.

2. ‘PP-1’ produces an abundance of flowers. Wing petals are Yellow-Orange Group 14D and Stand Petals transition from Orange Group 24C in the center to Yellow-Orange Group 14C toward the margins.

3. ‘PP-1’ produces a dense dark green canopy.

The new A. glabrata cultivar ‘PP-1’ can be compared to ‘Florigraze’, ‘Pointed Leaf’ and ‘Ecoturf’ (unpatented).

Plants of the new A. glabrata cultivar differ from ‘Florigraze’ in the following characteristics:

1. The new cultivar ‘PP-1’ produces more flowers than ‘Florigraze’.

2. The new cultivar ‘PP-1’ has a darker green plant color, maintains higher turf quality, is more dense, has narrower leaves, and has lower Pepper Spot ratings than ‘Florigraze’.

Plants of the new A. glabrata cultivar differ from ‘Pointed Leaf’ in the following characteristics:

1. The new cultivar ‘PP-1’ has narrower leaves and less leaflet area than ‘Pointed Leaf’.

Plants of the new A. glabrata cultivar differ from ‘Ecoturf’ in the following characteristics:

1. The new cultivar ‘PP-1’ maintains higher turf quality than ‘Ecoturf’.

2. Flower number ratings are significantly higher at most rating dates for ‘PP-1’ compared to ‘Ecoturf’.

3. ‘PP-1’ has significantly narrower leaves and significantly less total leaflet area then ‘Ecoturf’.

The following observations, measurements, and values describe plants grown in Tifton, Ga., Quincy, Fla. and Gainesville, Fla. In Tables 1-18, the least significant difference (LSD) is set at P<0.05 probability level. In 2006 we established ‘PP-1’ in a test at Tifton, Ga. (3.7×3.7 m plots, four replications) with the released or soon-to-be released perennial peanut cultivars from University of Florida. Dr. Kevin Kenworthy provided us planting materials of ‘Ecoturf’, ‘Arblick’, ‘Florigraze’, and ‘Arbrook’. Dr. Ann Blount established tests (1.5×3.1 m plots) at Quincy, Fla. (five replications) and at Gainesville, Fla. (four replications) with the same cultivars plus ‘Pointed Leaf’ at Quincy, Fla. (10 Apr. 2009) and at Gainesville, Fla. (28 June 2010). Dr. Mimi Williams, breeder of ‘Pointed Leaf’, provided us planting material of this cultivar in 2010. Eight replications of ‘Pointed Leaf’ and ‘PP-1’ were established as single plant propagules on 14 Jun. 2011 at Tifton, Ga. Plots at Tifton received 560 kg/ha 5-10-15 preplant and 280 kg/ha 5-10-15 yearly in April after establishment. Plots in Florida did not receive any fertilizer.

‘PP-1’ is a perennial, vegetatively propagated dark-green narrow-leaf perennial peanut recommended for use as an ornamental in USDA zones 8b and 9. It has survived in a 2006 replicated test at Tifton from 2006 to 2012 (when the test was destroyed). ‘PP-1’ was in the top statistical group for flower number on 10 of the 13 rating dates during 2008 to 2011 at Tifton (Table 1), in 3 of 5 rating dates at Quincy, Fla. in 2010 and 2011 (Table 11), and in 2 of 5 rating dates at Gainesville, Fla. in 2011 and 2012 (Table 12). PP-1 had significantly darker green color than the other cultivars at Tifton in 12 of 13 tests (Table 2). Mature leaf color was classified as Green 144A according to The Royal Horticultural Society color index (5^{th} Edition, 2007). ‘PP-1’ was in the top statistical group for color in 6 of 7 rating dates in the Florida tests (Table 13).

Height of ‘PP-1’ was similar to ‘Ecoturf’on 5 of 6 measuring dates in three years at Tifton, Ga. (Table 3). At most measuring dates it was shorter than ‘Arblick’ and ‘Arbrook’ and taller than ‘Florigraze’. At Quincy, Fla, ‘PP-1’, ‘Pointed Leaf’, and ‘Arblick’ were similar in height and shorter than ‘Ecoturf’, ‘Florigraze’, and ‘Arbrook’ (Table 14). ‘PP-1’ was rated in the top statistical group in four of five tests for turf quality (Table 4).

In the establishment year (2006) at Tifton, Ga., ‘PP-1’ and ‘Ecoturf’ were slower to establish than the other cultivars (Table 5). However, in subsequent years, ‘PP-1’ was in the top statistical group for maintaining dense ground cover. Canopy density at Tifton, Ga. (Table 6) was similar for ‘PP-1’, ‘Ecoturf’, and ‘Arblick’ and higher than ‘Florigraze’ and ‘Arbrook’ at most rating dates. Canopy density at Quincy, Fla. (Table 15) was similar to ‘Pointed Leaf’, ‘Arblick’, and ‘Florigraze’ at early and mid-season dates, but had less density than the other cultivars, except ‘Pointed Leaf’ in October due to pepper spot disease (Leptosphaerulina crassiasca).

Spring green-up at Gainesville, Fla. was similar for all cultivars, except for ‘Florigraze’ which showed a slower green-up (Table 16). ‘PP-1’ showed better freeze tolerance than ‘Ecoturf’ and ‘Arblick’ at Gainesville, Fla. and all of the cultivars tested at Quincy, Fla. (Table 16).

At Tifton, Ga., ‘PP-1’ had low Pepper Spot ratings at October and November rating dates (Table 7). The disease rating was higher at a December date in 2009, but less than the disease on ‘Florigraze’ and ‘Arbrook’. Pepper spot ratings in October were higher for ‘PP-1’ (and similar to ‘Pointed Leaf’) at Quincy, Fla. than at Tifton, Ga. (Table 17). Leaf scorch caused by the same organism as pepper spot was higher for ‘PP-1’ and ‘Pointed Leaf’ at Gainesville, Fla. than for the other cultivars in the test. Note: the higher disease ratings on ‘PP-1’ and ‘Pointed Leaf’ in the Florida tests and in the Tifton test in Dec. 2009 are probably due to fertility—mainly potassium. On 7 Oct. 2012, the ‘PP-1’ growing in the original collection area next to U.S. Route 41 showed high pepper spot infection while the adjacent fertilized 14 June 2011 test and a Foundation planting showed no disease symptoms. Similar leaf spot symptoms are observed on bermudagrass (Cynodon dactylon) in the fall due to the soil being depleted of potassium. No peanut stunt virus (Cucumovirus) was detected in ‘PP-1’ growing at Gainesville, Fla. in 2010, 2011 and 2012 (Table 18).

Morphologically, ‘PP-1’ is most similar to ‘Pointed Leaf’. However ‘PP-1’ has narrower leaves (Table 8) than ‘Pointed Leaf’, ‘A42’, and all of the cultivars tested. ‘PP-1’ and most of the cultivars tested have similar leaf length except ‘Arbrook’ which has longer leaves (Table 9). ‘PP-1’ has smaller leaves than all of the other cultivars except for ‘Florigraze’ on 25 Jul. 2011 at Tifton, Ga. and ‘Pointed Leaf’ on 21 Aug 2010 at Quincy, Fla, (Table 10).

A number of propagation studies have been conducted using rhizomes as sod (using a peanut inverter to dig the sod) and dug sprigs (using a traditional bermudagrass sprig harvester). Both propagation materials work well, but sprigs are easier to plant and handle. Best establishment takes place when sprigs are planted in furrows (continuous row of rhizomes with average density of 2 to 4 sprigs side by side), covered with 2.5 cm of soil. Rhizomes planted in lm wide rows establish in one year. Closer row spacings speed up establishment. A layer of rhizomes with 80% surface coverage planted in 23×46 cm flats establish as a marketable product in 5 to 6 weeks in the greenhouse.

TABLE 1a
Flower number ratings on five Arachis glabrata perennial
peanut genotypes planted at Tifton, GA on 18 May 2006.
Number of Flowers^{z}

| | 2008 | | | 2009 | | |
|---|---|---|---|---|---|---|
| Entry | May 1 | Jul. 1 | Sept. 2 | May 11 | Aug. 11 | Sept. 30 |
| ‘PP-1’ | 5.5 | 1.3 | 5.8 | 8.7 | 3.5 | 2.0 |
| ‘Ecoturf’ | 3.0 | 1.8 | 2.5 | 3.0 | 2.2 | 1.2 |
| ‘Arblick’ | 2.0 | 2.0 | 2.0 | 2.0 | 2.0 | 1.2 |
| ‘Florigraze’ | 2.3 | 2.0 | 2.0 | 2.5 | 2.0 | 1.7 |
| ‘Arbrook’ | 1.5 | 2.0 | 1.3 | 2.7 | 1.5 | 1.0 |
| LSD-5% | 0.8 | 0.6 | 0.5 | 0.8 | 0.9 | 0.5 |

^{z}1 = no flowers,
2 = less than 20% coverage,
3 = 20 to 30% coverage,
4 = 30 to 40% coverage,
5 = 40 to 50% coverage,
6 = 50 to 60% coverage,
7 = 60 to 70% coverage,
8 = 70 to 80% coverage,
9 = greater than 80% coverage.

TABLE 1b
Flower number ratings on five Arachis
glabrata perennial peanut genotypes
planted at Tifton, GA on 18 May 2006.
Number of Flowers^{z}

| | 2010 | | | 2011 | | | |
|---|---|---|---|---|---|---|---|
| | May | Sept. | Sept. | Jan. | Apr. | Apr. | Jul. |
| Entry | 15 | 1 | 29 | 20 | 19 | 25 | 27 |
| ‘PP-1’ | 7.0 | 5.5 | 5.0 | 2.0 | 2.7 | 6.5 | 7.2 |
| ‘Ecoturf’ | 4.2 | 3.0 | 2.7 | 2.7 | 2.0 | 2.0 | 3.5 |
| ‘Arblick’ | 4.0 | 4.0 | 2.5 | 2.7 | 1.7 | 2.2 | 2.7 |
| ‘Florigraze’ | 3.2 | 3.7 | 2.0 | 3.0 | 2.0 | 1.7 | 4.5 |
| ‘Arbrook’ | 2.0 | 1.7 | 1.0 | 2.2 | 1.5 | 2.0 | 2.0 |
| LSD-5% | 1.1 | 1.5 | 0.7 | NS | 1.7 | 1.7 | 1.1 |

^{z}1 = no flowers,
2 = less than 20% coverage,
3 = 20 to 30% coverage,
4 = 30 to 40% coverage,
5 = 40 to 50% coverage,
6 = 50 to 60% coverage,
7 = 60 to 70% coverage,
8 = 70 to 80% coverage,
9 = greater than 80% coverage.

TABLE 2a
Plant color ratings on five Arachis glabrata perennial peanut
genotypes planted at Tifton, GA 18 May 2006.

| | Color^{z} | | | | |
|---|---|---|---|---|---|
| | 2008 | | 2009 | | |
| Entry | 2 May | 7 Oct. | 11 Aug. | 30 Sept. | 16 Dec. |
| ‘PP-1’ | 7.0 | 8.0 | 8.0 | 9.0 | 9.0 |
| ‘Ecoturf’ | 6.0 | 6.0 | 5.5 | 6.2 | 6.7 |
| ‘Arblick’ | 7.0 | 7.0 | 6.0 | 7.2 | 8.0 |
| ‘Florigraze’ | 4.0 | 4.0 | 4.2 | 4.7 | 4.5 |
| ‘Arbrook’ | 8.0 | 7.0 | 4.2 | 5.7 | 5.5 |
| LSD-5% | 1.0 | 1.0 | 0.7 | 0.5 | 1.0 |

^{z}Color ratings are: 1 = yellow, 9 = dark green

TABLE 2b
Plant color ratings on five Arachis glabrata perennial
peanut genotypes planted at Tifton, GA on 18 May 2006.
Color^{z}

| | 2010 | | | | | 2011 | | |
|---|---|---|---|---|---|---|---|---|
| Entry | Apr. | Aug. 3 | Sept. 1 | Sept. 29 | Nov. 19 | Apr. 19 | Jun. 20 | Jul. 27 |
| ‘PP-1’ | 9.0 | 9.0 | 9.0 | 9.0 | 8.0 | 9.0 | 9.0 | 9.0 |
| ‘Ecoturf’ | 7.7 | 6.5 | 7.5 | 7.0 | 5.0 | 7.5 | 7.5 | 7.2 |
| ‘Arblick’ | 7.7 | 7.7 | 8.0 | 9.0 | 6.5 | 8.0 | 7.7 | 8.0 |
| ‘Florigraze’ | 6.2 | 4.0 | 6.2 | 5.5 | 3.5 | 6.2 | 6.5 | 6.0 |
| ‘Arbrook’ | 8.0 | 6.2 | 7.7 | 7.2 | 5.2 | 7.5 | 7.2 | 7.0 |
| LSD-5% | 0.5 | 0.5 | 0.7 | 0.8 | 0.7 | 0.5 | 0.8 | 0.7 |

^{z}Color ratings are: 1 = yellow, 9 = dark green

TABLE 3
Height of five Arachis glabrata perennial peanut
genotypes planted at Tifton, GA on 18 May 2006.
Height (cm)^{z}

| | 2008 | | | 2009 | 2011 | |
|---|---|---|---|---|---|---|
| Entry | May 2 | Jun. 2 | Jul. 1 | Oct. 15 | Apr. 19 | Jul. 27 |
| ‘PP-1’ | 6.0 | 8.0 | 10.0 | 18.5 | 13.0 | 19.5 |
| ‘Ecoturf’ | 6.0 | 8.0 | 11.0 | 20.5 | 5.7 | 18.7 |
| ‘Arblick’ | 8.0 | 9.0 | 13.0 | 30.0 | 10.2 | 25.0 |
| ‘Florigraze’ | 4.0 | 7.0 | 14.0 | 19.0 | 4.5 | 16.7 |
| ‘Arbrook’ | 14.0 | 16.0 | 17.0 | 27.0 | 14.0 | 23.7 |
| LSD-5% | 2.0 | 1.0 | 2.0 | 3.1 | 2.3 | 4.7 |

^{z}Plant height measured from ground level to top of plant canopy.

TABLE 4
Turf quality of five Arachis glabrata perennial peanut
genotypes planted at Tifton, GA on 18 May 2006.

| | Turf Quality^{z} | | | | |
|---|---|---|---|---|---|
| | 2008 | | | | |
| Entry | Jun. 2 | Jul. 7 | Aug. 1 | Sept. 2 | Oct. 7 |
| ‘PP-1’ | 6.5 | 9.0 | 8.0 | 9.0 | 6.8 |
| ‘Ecoturf’ | 6.0 | 7.0 | 7.8 | 7.3 | 5.8 |
| ‘Arblick’ | 7.8 | 7.8 | 8.0 | 8.0 | 6.8 |
| ‘Florigraze’ | 3.5 | 5.5 | 4.5 | 4.3 | 4.8 |
| ‘Arbrook’ | 4.0 | 3.8 | 5.3 | 2.5 | 6.3 |
| LSD-5% | 1.1 | 0.9 | 0.7 | 1.0 | 0.8 |

^{z}Visual turf quality (based on color, density, and texture) rating on scale of 1 to 9 with 1 = poor and 9 = excellent. A rating of 6 is considered acceptable turf quality.

TABLE 5
Percent ground cover on five Arachis glabrata perennial
peanut genotypes planted at Tifton, GA on 18 May 2006.

| | % Cover^{z} | | | | |
|---|---|---|---|---|---|
| | 2006 | 2009 | 2010 | 2011 | |
| Entry | Oct. 20 | Apr. 9 | Apr. 12 | Apr. 19 | Jan. 20 |
| ‘PP-1’ | 42.0 | 97.5 | 100.0 | 9.0 | 9.0 |
| ‘Ecoturf’ | 57.0 | 70.0 | 95.0 | 5.2 | 7.0 |
| ‘Arblick’ | 75.0 | 87.5 | 97.0 | 6.7 | 8.5 |
| ‘Florigraze’ | 77.0 | 10.0 | 77.0 | 2.0 | 5.2 |
| ‘Arbrook’ | 72.0 | 35.0 | 57.0 | 4.0 | 7.0 |
| LSD-5% | 17.0 | 11.7 | 11.0 | 1.6 | 2.1 |

^{z}1 = less than 20% canopy coverage,
2 = 20 to 30% canopy coverage,
3 = 30 to 40% canopy coverage,
4 = 40 to 50% canopy coverage,
5 = 50 to 60% canopy coverage,
6 = 60 to 70% canopy coverage,
7 = 70 to 80% canopy coverage,
8 = 80 to 90% canopy coverage,
9 = 100% canopy coverage.

TABLE 6
Canopy density ratings on five Arachis glabrata perennial
peanut genotypes planted at Tifton, GA 18 May 2006.

| | Density^{z} | | | | | | |
|---|---|---|---|---|---|---|---|
| | 2008 | 2009 | 2010 | | | | 2011 |
| | Oct. | Oct. | Aug. | Sept. | Sept. | Nov. | Jul. |
| Entry | 7 | 15 | 3 | 1 | 29 | 29 | 27 |
| ‘PP-1’ | 8.0 | 8.0 | 9.0 | 9.0 | 9.0 | 8.0 | 9.0 |
| ‘Ecoturf’ | 7.0 | 8.7 | 7.2 | 8.7 | 9.0 | 7.2 | 7.5 |
| ‘Arblick’ | 7.8 | 8.5 | 7.2 | 9.0 | 8.7 | 7.7 | 8.2 |
| ‘Florigraze’ | 4.5 | 4.2 | 4.7 | 6.7 | 6.5 | 3.5 | 6.0 |
| ‘Arbrook’ | 5.8 | 6.0 | 5.2 | 6.7 | 6.5 | 4.7 | 6.7 |
| LSD-5% | 1.1 | 1.0 | 1.0 | 0.5 | 0.9 | 0.7 | 0.8 |

^{z}1 = less than 20% canopy coverage,
2 = 20 to 30% canopy coverage,
3 = 30 to 40% canopy coverage,
4 = 40 to 50% canopy coverage,
5 = 50 to 60% canopy coverage,
6 = 60 to 70% canopy coverage,
7 = 70 to 80% canopy coverage,
8 = 80 to 90% canopy coverage,
9 = 100% canopy coverage.

TABLE 7
Pepper spot ratings on five Arachis glabrata perennial
peanut genotypes planted at Tifton, GA on 18 May 2006.

| | Disease- Pepper Spot^{z} | | | |
|---|---|---|---|---|
| | 2008 | 2009 | | 2010 |
| Entry | Oct. 7 | Oct. 15 | Dec. 16 | Nov. 29 |
| ‘PP-1’ | 1.0 | 1.0 | 3.5 | 1.5 |
| ‘Ecoturf’ | 1.3 | 2.0 | 3.2 | 2.2 |
| ‘Arblick’ | 1.0 | 1.0 | 2.0 | 1.7 |
| ‘Florigraze’ | 1.5 | 6.0 | 5.7 | 4.2 |
| ‘Arbrook’ | 1.0 | 4.2 | 5.0 | 4.2 |
| LSD-5% | 0.4 | 1.3 | 1.0 | 2.0 |

^{z}Pepper spot- 1 = no disease, 9 = high disease. Disease caused by Leptosphaeeruhna crassiasca.

TABLE 8
Leaf width on individual leaves of eight Arachis glabrata
perennial peanut genotypes planted at Tifton, GA and Quincy, FL.
Leaf Width (mm)

| | Tifton, GA | | | | | Quincy, FL |
|---|---|---|---|---|---|---|
| | 2009 | | 2011 | 2012 | | 2010 |
| Entry | Jul. 30^{z} | Aug. 31^{y} | Jul. 25^{x} | Jul. 25^{z} | May 29^{y} | Aug. 31^{w} |
| ‘PP-1’ | 4.0 | 6.9 | 7.5 | 6.0 | 6.7 | 7.8 |
| ‘Pointed Leaf’ | | 10.0 | | | 8.5 | 8.4 |
| ‘Ecoturf’ | 10.0 | | | 11.0 | | |
| ‘Arblick’ | 10.0 | | | 11.0 | | |
| ‘Florigraze’ | 8.0 | | | 8.0 | | |
| ‘Arbrook’ | 11.0 | | | 10.0 | | |
| A42 | | | 8.8 | | | |
| LSD-0.05% | 1.0 | 0.5 | 1.2 | 1.0 | 0.9 | 0.6 |

^{z}Planted 18 May 2006 at Tifton, GA. Four replications per entry. Six leaflets were measured per replication.
^{y}Planted 14 Jun. 2011 at Tifton, GA. Seven replications per entry. Three leaflets were measured per replications.
^{x}Planted 26 May 2010. Ten replications per entry. Three leaflets were measured per
replication.
^{w}Planted 10 Apr. 2009 Quincy, FL. Five replications per entry. Six leaflets were measured per replication.

TABLE 9
Leaf length on individual leaves of eight Arachis glabrata genotypes
planted on 18 May 2006 and 2010 at Tifton, GA and Quincy, FL.
Leaf Length (mm)

| | Tifton, GA | | | | | Quincy, FL |
|---|---|---|---|---|---|---|
| | 2009 | | 2011 | 2012 | | 2010 |
| Entry | Jul. 30^{z} | Aug. 31^{y} | Jul. 25^{x} | Jul. 25^{z} | May 29^{y} | Aug. 21^{w} |
| ‘PP-1’ | 25.0 | 31.6 | 34.3 | 22.0 | 30.2 | 36.6 |
| ‘Pointed Leaf’ | | 27.5 | | | 27.8 | 35.5 |
| ‘Ecoturf’ | 26.0 | | | 18.0 | | |
| ‘Arblick’ | | | | 17.0 | | |
| ‘Florigraze’ | 28.0 | | | 18.0 | | |
| ‘Arbrook’ | 36.0 | | | 21.0 | | |
| A42 | | | 38.1 | | | |
| LSD-0.05% | 3.0 | 4.1 | 5.0 | 2.0 | 2.0 | 2.7 |

^{z}Planted 18 May 2006 at Tifton, GA. Four replications per entry. Six leaflets were measured per replication.
^{y}Planted 14 Jun. 2011 at Tifton, GA. Seven replications per entry. Three leaflets were measured per replications.
^{x}Planted 26 May 2010. Ten replications per entry. Three leaflets were measured per
replication.
^{w}Planted 10 Apr. 2009 Quincy, FL. Five replications per entry. Six leaflets were measured per replication.

TABLE 10
Leaf area on individual leaves of eight Arachis glabrata
genotypes planted at Tifton, GA and Quincy, FL.
Leaf Area (cm²)

| | Tifton, GA | | | | | Quincy, FL |
|---|---|---|---|---|---|---|
| | 2009 | | 2011 | 2012 | | 2010 |
| Entry | Jul. 30^{z} | Aug. 31^{y} | Jul. 25^{x} | Jul. 25^{z} | May 29^{y} | Aug. 21 |
| ‘PP-1’ | 3.0 | 4.6 | 5.2 | 4.0 | 4.2 | 5.1 |
| ‘Pointed Leaf’ | | 6.5 | | | 5.3 | 5.5 |
| ‘Ecoturf’ | 7.0 | | | 6.4 | | |
| ‘Arblick’ | 6.0 | | | 6.1 | | |
| ‘Florigraze’ | 6.0 | | | 4.7 | | |
| ‘Arbrook’ | 11.0 | | | 6.7 | | |
| A42 | | | 7.4 | | | |
| LSD-0.05% | 2.0 | 1.0 | 1.5 | 1.0 | 0.7 | 0.8 |

^{z}Planted 18 May 2006 at Tifton, GA. Four replications per entry. Six leaflets were measured per replication.
^{y}Planted 14 Jun. 2011 at Tifton, GA. Seven replications per entry. Three leaflets were measured per replications.
^{x}Planted 26 May 2010. Ten replications per entry. Three leaflets were measured per
replication.
^{w}Planted 10 Apr. 2009 Quincy, FL. Five replications per entry. Six leaflets were measured per replication.

TABLE 11
Flower number ratings on six Arachis glabrata perennial
peanut genotypes planted at Quincy, FL on 10 Apr. 2009.

| | Ratings for Number of Flowers^{z} | | | | |
|---|---|---|---|---|---|
| | 2010 | | | 2011 | |
| Cultivar | 2 Feb. | 30 Jul. | 30 Jul.^{y} | 1 Jun. | 1 Jun.^{x} |
| ‘PP-1’ | 4.8 | 4.2 | 5.6 | 3.8 | 3.0 |
| ‘Pointed Leaf’ | 4.8 | 5.0 | 6.0 | 4.0 | 3.2 |
| ‘Ecoturf’ | 2.0 | 2.4 | 2.8 | 2.6 | 4.0 |
| ‘Arblick’ | 4.6 | 5.0 | 5.8 | 5.2 | 6.0 |
| ‘Florigraze’ | 3.8 | 3.4 | 3.6 | 2.8 | 2.8 |
| ‘Arbrook’ | 3.2 | 2.0 | 2.0 | 2.4 | 1.6 |
| LSD - 5% | 0.7 | 0.9 | 0.8 | 1.5 | 1.4 |

^{z}Rating for number of flowers on scale of 1 to 5 with 1 = none and 5 = many.
^{y}After defoliation to six cm on 14 Jul., 2010.
^{x}After defoliation to six cm on 12 May, 2011.

TABLE 12
Flower number ratings on six Arachis glabrata perennial
peanut genotypes planted at Gainesville, FL on Jun. 28, 2010.

| | Flower Number^{z} | | | | |
|---|---|---|---|---|---|
| | 2011 | | | | 2012 |
| Cultivar | 20 Apr. | 1 Jun. | 30 Sept. | 22 Oct. | 21 Mar. |
| ‘PP-1’ | 1.0 | 4.0 | 3.8 | 2.5 | 4.2 |
| ‘Pointed Leaf’ | 1.0 | 3.7 | 3.2 | 2.2 | 2.7 |
| ‘Ecoturf’ | 1.5 | 4.5 | 2.0 | 3.2 | 2.5 |
| ‘Arblick’ | 1.7 | 6.0 | 3.8 | 3.7 | 1.0 |
| ‘Florigraze’ | 2.5 | 5.2 | 2.2 | 4.2 | 2.7 |
| ‘Arbrook’ | 1.7 | 3.5 | 1.0 | 2.5 | 1.7 |
| LSD - 5% | 0.8 | 2.0 | 0.9 | 0.7 | 1.2 |

^{z}Flower number ratings on scale of 1 to 9 where 1 = no flowers and 9 = profuse flowering.

TABLE 13
Ratings for plant color on six Arachis
glabrata perennial peanut genotypes
planted at Gainesville, FL^{z} and Quincy, FL^{y}.
Plant Color^{x}

| | Quincy | | | | Gainesville | | |
|---|---|---|---|---|---|---|---|
| | 2010 | | | | 2011 | | |
| | 30 | 30 | 27 | 27 | 20 | 15 | 22 |
| Entry | Jul. | Jul.^{w} | Oct. | Oct.^{w} | Apr. | Jun. | Oct. |
| ‘PP-1’ | 8.6 | 9.0 | 6.6 | 7.2 | 2.7 | 2.7 | 2.2 |
| ‘Pointed | 8.8 | 8.8 | 7.0 | 7.4 | 2.5 | 3.0 | 2.0 |
| Leaf’ | | | | | | | |
| ‘Ecoturf’ | 7.6 | 7.6 | 6.6 | 7.8 | 2.0 | 2.5 | 2.0 |
| ‘Arblick’ | 7.6 | 7.8 | 6.0 | 7.2 | 2.0 | 3.0 | 2.0 |
| ‘Florigraze’ | 6.4 | 6.4 | 5.0 | 6.4 | 2.0 | 1.2 | 2.0 |
| ‘Arbrook’ | 7.8 | 8.0 | 6.0 | 7.0 | 2.0 | 2.0 | 2.0 |
| LSD-5% | 0.6 | 0.9 | 1.0 | 0.8 | 0.5 | 0.6 | 0.3 |

^{z}Planted Jun. 28, 2010.
^{y}Planted Apr. 10, 2009.
^{x}Plant color ratings on scale of 1 to 9 where 1 = brown and 9 = dark green.
^{w}After defoliation to six cm on Jul. 14, 2010.

TABLE 14
Height measurements on six Arachis glabrata perennial
peanut genotypes planted at Quincy, FL on Apr. 10, 2009.
Height (cm)^{z}

| | 2010 | | | | 2011 | |
|---|---|---|---|---|---|---|
| Cultivar | 30 Jul. | 30 Jul.^{y} | 27 Oct. | 27 Oct.^{y} | 1 Jun. | 1 Jun.^{x} |
| ‘PP-1’ | 16 | 10 | 14 | 9 | 14 | 9 |
| ‘Pointed Leaf’ | 11 | 9 | 13 | 8 | 11 | 10 |
| ‘Ecoturf’ | 35 | 17 | 27 | 18 | 29 | 13 |
| ‘Arblick’ | 15 | 13 | 15 | 13 | 14 | 6 |
| ‘Florigraze’ | 36 | 24 | 24 | 26 | 30 | 27 |
| ‘Arbrook’ | 39 | 38 | 30 | 39 | 43 | 43 |
| LSD-5% | 6 | 4 | 7 | 4 | 5 | 5 |

^{z}Height measured from ground level to top of canopy.
^{y}After defoliation to six cm on Jul. 14, 2010.
^{x}After defoliation to six cm on May 12, 2011.

TABLE 15
Rating for canopy density on six Arachis glabrata perennial
peanut genotypes planted at Quincy, FL on Apr. 10, 2009.

| | Canopy Density^{z} | | | | |
|---|---|---|---|---|---|
| | 2010 | | | 2011 | |
| Cultivar | 30 Jul. | 30 Jul.^{z} | 27 Oct. | 27 Oct.^{y} | 1 Jun. |
| ‘PP-1’ | 7.2 | 7.2 | 4.0 | 4.0 | 8.6 |
| ‘Pointed Leaf’ | 6.4 | 7.0 | 3.2 | 4.0 | 8.4 |
| ‘Ecoturf’ | 7.4 | 8.4 | 7.4 | 8.4 | 8.8 |
| ‘Arblick’ | 7.6 | 7.8 | 7.2 | 8.0 | 9.0 |
| ‘Florigraze’ | 6.4 | 7.8 | 4.8 | 7.2 | 9.0 |
| ‘Arbrook’ | 5.6 | 6.2 | 5.2 | 6.4 | 8.6 |
| LSD - 5% | 1.0 | 0.8 | 1.0 | 0.7 | 0.6 |

^{z}Canopy density ratings on scale of 1 to 9 where 1 = bare ground and 9 = full canopy.
^{y}After defoliation to six cm on 14 Jul., 2010.

TABLE 16
Ratings for spring green-up and freeze damage
(on leaves) on six Arachis glabrata perennial peanut
genotypes planted at Gainesville, FL^{z} and Quincy, FL^{y}.

| | Spring Green-up^{x} | Freeze Damage^{w} | |
|---|---|---|---|
| Cultivar | Mar. 21, 2012 | Nov. 18, 2011 | Jan. 5, 2012 |
| ‘PP-1’ | 4.9 | 7.5 | 4.6 |
| ‘Pointed | 5.0 | 10.0 | 3.4 |
| Leaf’ | | | |
| ‘Ecoturf’ | 5.0 | 45.0 | 3.2 |
| ‘Arblick’ | 5.0 | 51.2 | 3.0 |
| ‘Florigraze’ | 3.5 | 40.0 | 3.2 |
| ‘Arbrook’ | 4.7 | 25.5 | 3.6 |
| LSD - 5% | 0.5 | 34.9 | 0.6 |

^{z}Planted Jun. 28, 2010.
^{y}Planted Apr. 10, 2009.
^{x}Spring green-up at Gainesville, FL rated on a scale of 1 to 5 where 1 = brown and 5 = completely green.
^{w}Freeze damage to leaves at Gainesville, FL on Nov. 18, 2011 rated as percent leaf damage after −2° C. for two days.
Freeze damage ratings on leaves on scale of 1 to 5 at Quincy, FL on Jan. 5, 2012 where 1 = severe damage and 5 = tolerant. Rated two days after −8° C. for two days.

TABLE 17
Leaf scorch and leaf spot ratings on six Arachis glabrata perennial
peanut genotypes planted at Quincy, FL on Apr. 10, 2009.

| | Leaf Scorch^{z} | | | | | Leaf Spot^{z} | | | | |
|---|---|---|---|---|---|---|---|---|---|---|
| | 2010 | | 2011 | | 2012 | 2010 | | 2011 | | 2012 |
| Cultivar | 15 Jul. | 27 Oct. | 1 Jun. | 20 Oct. | 1 Jun. | 15 Jul. | 27 Oct. | 1 Jun. | 10 Oct. | 1 Jun. |
| ‘PP-1’ | 4.2 | 3.0 | 3.4 | 3.4 | 3.6 | 1.0 | 1.0 | 1.0 | 1.0 | 1.0 |
| ‘Pointed Leaf’ | 4.4 | 2.8 | 3.0 | 3.6 | 3.4 | 1.0 | 1.0 | 1.0 | 1.0 | 1.0 |
| ‘Ecoturf’ | 1.4 | 1.0 | 1.0 | 1.0 | 1.0 | 1.0 | 1.0 | 1.0 | 1.0 | 1.0 |
| ‘Arblick’ | 1.0 | 1.0 | 1.0 | 1.0 | 1.0 | 1.0 | 1.0 | 1.0 | 1.0 | 1.0 |
| ‘Florigraze’ | 1.2 | 1.6 | 1.0 | 2.2 | 2.0 | 8.4 | 6.6 | 7.4 | 7.2 | 6.2 |
| ‘Arbrook’ | 1.0 | 1.0 | 1.0 | 1.0 | 1.0 | 2.6 | 2.2 | 2.2 | 2.0 | 2.0 |
| LSD-5% | 0.6 | 0.4 | 0.7 | 0.4 | 0.4 | 0.4 | 0.4 | 0.4 | 0.5 | 0.2 |

^{z}Pepper spot and leaf scorch ratings on scale of 1= no disease, 5 = severe lesions and 9 = leaf drop. Both diseases caused by Leptosphaerulina crassiasca.

In summary, ‘PP-1’ is a dark-green, narrow-leafed plant that produces an abundance of yellow orange flowers in the spring, summer and fall. It has performed as well or better in GA and FL than most released cultivars. It has performed well in Poteet, Tex. and Lake City, Fla., in non-replicated tests and in yards at Brian Schwartz's and Wayne Hanna's homes for two to six years. ‘PP-1’ produces a low-maintenance, low-input, ornamental, and colorful ground cover. Preliminary studies show that it can be used in combination with lawn grasses to furnish nitrogen for the grass.

### BRIEF DESCRIPTION OF THE FIGURES

The accompanying colored photographs illustrate the overall appearance and distinct characteristics of the new cultivar of A. glabrata, ‘PP-1’. The colors in the photographs are as close as possible with the photographic and printing technology utilized.

FIG. 1 is a photograph of the new cultivar of A. glabrata, ‘PP-1’. FIG. 2 is a closeup photograph of a flower and leaves.

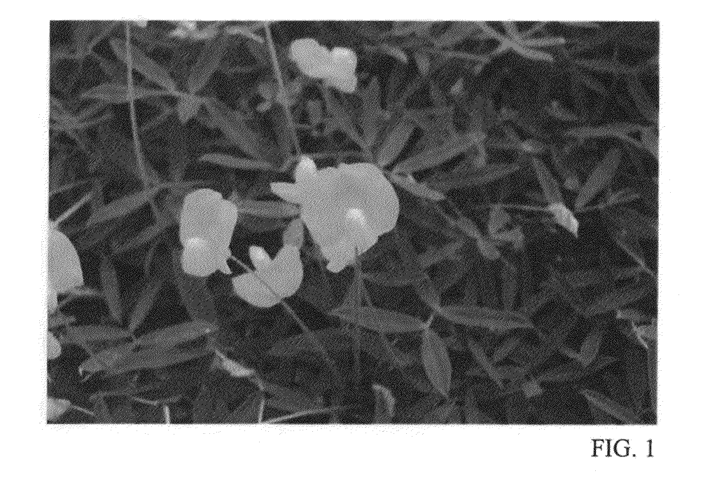

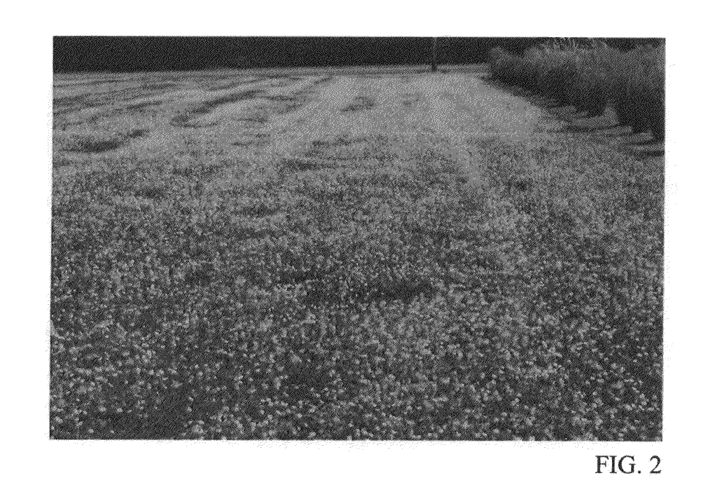

### BOTANICAL DESCRIPTION

‘PP-1’ is a perennial, vegetatively propagated dark-green narrow-leaf perennial peanut recommended for use as an ornamental in USDA zones 8b and 9. It has survived in a 2006 replicated test at Tifton from 2006 to 2012 (when the test was destroyed). It has survived under no management next to perennial peanut research plot area since 1954. All data are from plants established as single stem propagules in mid-May, and rated throughout the years.
Plant:
   Mature plant height.—Approximately 6-20 cm.
   Leaf width.—Approximately 4-8 mm.
   Leaf length.—Approximately 27-32 mm.
   Adaxial leaf color.—About Green RHS 144A.
   Abaxial leaf color About Green RHS 144A.
   Standard petal flower color.—Stand Petals transition from Orange Group 24C in the center to Yellow-Orange Group 14C toward the margins.
   Wing petal flower color.—Yellow-Orange Group 14D.
   Cucumvorus peanut stunt virus.—None.

What is claimed is:

1. A new and distinct cultivar of the Arachis glabrata plant named ‘PP-1’ substantially as illustrated and described herein.
